(19) United States
(12) Patent Application Publication
(10) Pub. No.: US 2022/0031585 A1
(43) Pub. Date: Feb. 3, 2022

(54) COSMETIC COMPOSITION FOR SCULPTING NAILS, METHOD THEREOF, AND A BRUSH-IN BOTTLE

(71) Applicant: Revlon Consumer Products Corporation, New York, NY (US)

(72) Inventors: Thong Vu, Vista, CA (US); Yinjung Lee, Vista, CA (US); Daniel Moore, Escondido, CA (US); Diane Larsen, Escondido, CA (US)

(21) Appl. No.: 16/944,971

(22) Filed: Jul. 31, 2020

(51) Int. Cl. A61K 8/25; A61K 8/04; A61Q 3/02; A61K 8/81; A61K 8/55; A45D 31/00; A45D 40/26

(52) U.S. Cl. CPC A61K 8/25; A61K 8/042; A61Q 3/02; A61K 8/8147; A61K 8/55; A61K 2800/81; A45D 40/262; A61K 2800/43; A61K 2800/26; A61K 2800/87; A45D 31/00

(57) ABSTRACT

A cosmetic composition for sculpting nails, a method for sculpting nails which comprises the application of the cosmetic composition, and a brush-in bottle for applying the cosmetic composition are disclosed.

### FIELD

The present disclosure relates to a cosmetic composition for sculpting nails, a method for sculpting a nail, which comprises the application of the cosmetic composition, and a brush-in bottle for applying the cosmetic composition.

### BACKGROUND

Sculpting gels are used to build artificial nail enhancements, which increase nail thickness and/or nail length. The gel is applied to a level that is thicker than that of regular polish or a gel polish. In order to sculpt nail gel, the applicator needs to have knowledge about how to form the enhancement with the proper structure, so that the artificial nail can last through the variety of physical stresses during wear. Obtaining an “apex” at the stress point is known to help the sculpted nail be less likely to crack or break at this stress point.

Because of gravity, the thickness of the wet gel tends to lose its shape before the gel gets a chance to be cured. When that thick layer flattens, it expands its perimeter beyond where the applicator has applied it. As a consequence, the gel runs towards and touches the skin or the cuticle. Without proper cleaning before curing the gel, skin or cuticle exposure occurs, and the chance of the nail coating being peeled off from the nail plate increases.

The cosmetic nail industry has considered different approaches in order to prevent sagging of the gel. Two similar approaches consist of increasing the viscosity of the gel, or increasing the viscosity and thixotropy of the gel, and in both approaches the gels are packaged in pots or jars. However, in order to reduce sag significantly, the viscosity of the sculpting gel needs to be significantly high, which reduces its workability, making it more sticky and stringy during application. Furthermore, the runniness of the gel is low, such that the gel does not self-level quickly, which results in the top surface not being smooth and instead having a wavy profile. Then, nail professionals have to file the cured sculpted nail to smooth out the surface, which is time consuming and may lead to job injuries, such as carpal tunnel syndrome, over the years.

Another approach for sculpting nails consists of a thin viscosity glue in a brush-in bottle and a powder in a jar. The thin viscosity glue is thinly painted on the nail, and then, the nail is dipped into the powder. “Dip” systems are preferred by nail professionals because conveniently require little effort by painting, as opposed to sculpting. The physical properties of the “dip” systems are quite similar to that of sculpting gels. However, in order to obtain sculpted nails, the sequence of painting and dipping steps has to be repeated multiple times. Moreover, dipping the nail into the powder is not a clean process because some powder particles flow in the air and drop onto nearby surfaces when the nail is withdrawn from the jar.

Therefore, there is still the need in the art for sculptable nail gels that can be applied with little effort, i.e. painting like a gel polish, with few repeating steps, and without using powder. Gel polishes cannot fulfill this need because gel polishes are not sculptable.

### SUMMARY

In a first aspect, a disclosed composition is a cosmetic composition comprising at least one hydrophobic fumed silica and at least one hydrophilic fumed silica, wherein the cosmetic composition is a gel sculpting nail composition.

In another aspect, a disclosed method is a method of sculpting an artificial nail onto at least a portion of a nail, the method comprising: applying onto at least a portion of the nail a layer of a cosmetic composition comprising at least one hydrophobic fumed silica and at least one hydrophilic fumed silica, wherein the cosmetic composition is a gel; sculpting the cosmetic composition into a desired shape and length; and curing the cosmetic composition.

In a third aspect, a disclosed bottle is a brush-in bottle that contains a cosmetic composition comprising at least one hydrophobic fumed silica and at least one hydrophilic fumed silica, wherein the cosmetic composition is a gel sculpting nail composition applied or applicable to at least a portion of a nail with the brush.

### DETAILED DESCRIPTION

Embodiments of the present invention are discussed in detail below. In describing embodiments, specific terminology is employed for the sake of clarity. However, the invention is not intended to be limited to the specific terminology so selected. While specific exemplary embodiments are discussed, it should be understood that this is done for illustration purposes only. A person skilled in the relevant art will recognize that other components and configurations can be used without departing from the spirit and scope of the invention. While a number of embodiments and features are described herein, it is to be understood that the various features of the invention and aspects of embodiments, even if described separately, may be combined unless mutually exclusive or contrary to the specific description. All references cited herein are incorporated by reference as if each had been individually incorporated.

As used herein, the transitional term “comprising,” which is synonymous with “including,” “containing,” or “characterized by,” is inclusive or open-ended and does not exclude additional, un-recited elements or method steps. However, in each recitation of “comprising” herein, it is intended that the term also encompass, as alternative embodiments, the phrases “consisting essentially of” and “consisting of,” where “consisting of,” excludes any element or step not specified and “consisting essentially of” permits the inclusion of additional un-recited elements or steps that do not materially affect the essential or basic and novel characteristics of the composition or method under consideration.

The present disclosure relates to a cosmetic composition comprising at least one hydrophobic fumed silica and at least one hydrophilic fumed silica, wherein the cosmetic composition is a gel sculpting nail composition.

This gel sculpting nail composition has a viscosity thin enough to be packaged like a gel polish, such as in a bottle. The gel sculpting nail composition has a viscosity thin enough to be applied to the nail like a gel polish, i.e. by painting, without sagging and without the addition of powders. Furthermore, the gel sculpting nail composition of the present disclosure can be applied with a thickness which allows a low number of layers to be applied, and therefore, saves time when sculpting nails.

In this disclosure “nail” and “nail surface” mean the natural, keratinaceous surface protecting the upper end of each finger and toe of humans, to which the cosmetic composition of the present disclosure is directly applied, or a polymerizable base coat composition is applied, and upon which the cosmetic composition of the present disclosure is applied.

In the present disclosure, “sculpt” or “sculpting” is understood as the formation of an artificial nail of a desired shape and length upon a portion of or the whole nail surface, and optionally upon a plastic, paper, or cardboard fingernail extension form joined to the finger, by polymerization of the cosmetic composition of the present disclosure, such as by means of a nail lamp.

In this disclosure, “gel” is a colloid semi-solid form with a jellylike texture that slightly flows due to gravity when in the steady-state.

In the present disclosure, “fumed silica”, also called “pyrogenic silica”, is understood as a silica produced under a flame resulting in silica particles with a low bulk density and high surface area. The surface area of the fumed silicas of the present disclosure ranges from 50 to 600 m²/g, from 100 to 300 m²/g, from 150 to 250 m²/g. The bulk density ranges from 1 kg/m³ to 400 kg/m³, from 10 kg/m³ to 300 kg/m³, from 25 kg/m³ to 200 kg/m³.

In this disclosure “hydrophobic fumed silica” is understood as a fumed silica where hydrophobic groups have been bonded with hydrogen bonds to the silica surface. Hydrophobic fumed silica cannot be dispersed in water. Hydrophobic groups bonded to the silica surface are, for example, alkyl or polydimethylsiloxane groups, like dimethicone, dimethyl silyl, trimethyl silyl, cetyl trimethoxysilyl, and capryl trimethoxysilyl. International Nomenclature of Cosmetic Ingredients (INCI) names for hydrophobic fumed silica are, for example, Silica Dimethicone Silylate, Silica Dimethyl Silylate, Silica Silylate, Silica Cetyl Silylate, or Silica Capryl Silylate. In some implementations, a hydrophobic fumed silica may provide structure or integrity to the disclosed compositions, such as to make them thicker or less runny. A hydrophobic fumed silica may make the disclosed compositions cloudy or opaque.

In the present disclosure “hydrophilic fumed silica” is understood as a fumed silica the surface of which has not been treated. Hydrophilic fumed silica is moistened with water and can be dispersed in water. Hydrophilic fumed silica falls under the INCI name “Silica” although not all the ingredients with the INCI name “Silica” are hydrophilic fumed silicas. In some implementations, a hydrophilic fumed silica may help produce a clear composition, including after curing the composition. A composition that is clear when cured may look like a natural nail or may be painted with a light-colored polish without affecting the apparent color of the polish.

In the cosmetic composition of the present disclosure, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the hydrophobic fumed silica may have the INCI name Silica Dimethicone Silylate, Silica Dimethyl Silylate, Silica Silylate, Silica Cetyl Silylate, or Silica Capryl Silylate.

The at least one hydrophobic fumed silica may be present in an amount between 0.1% and 6%, between 0.2% and 4.0%, between 0.3% and 2.5%, between 0.4% and 2.0% by weight of the total weight of the cosmetic composition. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one hydrophobic fumed silica may be present in an amount between 0.1% and 6%, between 0.2% and 4.0%, between 0.3% and 2.5%, between 0.4% and 2.0% by weight of the total weight of the cosmetic composition.

The at least one hydrophobic fumed silica may be present in an amount between 0.1% and 6%, between 0.2% and 4.0%, between 0.3% and 2.5%, between 0.4% and 2.0% by weight of the total weight of the cosmetic composition, and the hydrophobic fumed silica may have the INCI name Silica Dimethicone Silylate, Silica Dimethyl Silylate, Silica Silylate, Silica Cetyl Silylate, or Silica Capryl Silylate. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one hydrophobic fumed silica may be present in an amount between 0.1% and 6%, between 0.2% and 4.0%, between 0.3% and 2.5%, between 0.4% and 2.0% by weight of the total weight of the cosmetic composition, and the hydrophobic fumed silica may have the INCI name Silica Dimethicone Silylate, Silica Dimethyl Silylate, Silica Silylate, Silica Cetyl Silylate, or Silica Capryl Silylate.

The at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, and the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one hydrophobic fumed silica may be present in an amount between 0.1% and 6%, between 0.2% and 4.0%, between 0.3% and 2.5%, between 0.4% and 2.0% by weight of the total weight of the cosmetic composition, and the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition.

The at least one hydrophilic fumed silica may be present in an amount between 1.75% and 3.0% by weight of the total weight of the cosmetic composition. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one hydrophilic fumed silica may be present in an amount between 1.75% and 3.0% by weight of the total weight of the cosmetic composition. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, and the at least one hydrophilic fumed silica may be present in an amount between 1.75% and 3.0% by weight of the total weight of the cosmetic composition. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one hydrophobic fumed silica may be present in an amount between 0.1% and 6%, between 0.2% and 4.0%, between 0.3% and 2.5%, between 0.4% and 2.0% by weight of the total weight of the cosmetic composition, and the at least one hydrophilic fumed silica may be present in an amount between 1.75% and 3.0% by weight of the total weight of the cosmetic composition.

The at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate.

The at least one hydrophilic fumed silica may be present in an amount between 1.75% and 3.0% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one hydrophilic fumed silica may be present in an amount between 1.75% and 3.0% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 1.75% and 3.0% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 1.75% and 3.0% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate.

The sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, and the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition. The at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, and the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, and the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, and the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition.

The sum of the at least one hydrophobic fumed silica and the at least one hydrophilic fumed silica may range from 2.2% to 4.2% by weight of the total weight of the cosmetic composition. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, and the sum of the at least one hydrophobic fumed silica and the at least one hydrophilic fumed silica may range from 2.2% to 4.2% by weight of the total weight of the cosmetic composition. The at least one hydrophilic fumed silica may be present in an amount between 1.75% and 3.0% by weight of the total weight of the cosmetic composition, and the sum of the at least one hydrophobic fumed silica and the at least one hydrophilic fumed silica may range from 2.2% to 4.2% by weight of the total weight of the cosmetic composition. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the sum of the at least one hydrophobic fumed silica and the at least one hydrophilic fumed silica may range from 2.2% to 4.2% by weight of the total weight of the cosmetic composition. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 1.75% and 3.0% by weight of the total weight of the cosmetic composition, and the sum of the at least one hydrophobic fumed silica and the at least one hydrophilic fumed silica may range from 2.2% to 4.2% by weight of the total weight of the cosmetic composition. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 1.75% and 3.0% by weight of the total weight of the cosmetic composition, and the sum of the at least one hydrophobic fumed silica and the at least one hydrophilic fumed silica may range from 2.2% to 4.2% by weight of the total weight of the cosmetic composition.

The sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate.

The sum of the at least one hydrophobic fumed silica and the at least one hydrophilic fumed silica may range from 2.2% to 4.2% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the sum of the at least one hydrophobic fumed silica and the at least one hydrophilic fumed silica may range from 2.2% to 4.2% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one hydrophilic fumed silica may be present in an amount between 1.75% and 3.0% by weight of the total weight of the cosmetic composition, the sum of the at least one hydrophobic fumed silica and the at least one hydrophilic fumed silica may range from 2.2% to 4.2% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the sum of the at least one hydrophobic fumed silica and the at least one hydrophilic fumed silica may range from 2.2% to 4.2% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 1.75% and 3.0% by weight of the total weight of the cosmetic composition, the sum of the at least one hydrophobic fumed silica and the at least one hydrophilic fumed silica may range from 2.2% to 4.2% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 1.75% and 3.0% by weight of the total weight of the cosmetic composition, the sum of the at least one hydrophobic fumed silica and the at least one hydrophilic fumed silica may range from 2.2% to 4.2% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate.

The cosmetic composition of the present disclosure may comprise at least one reactive monomer, at least one reactive oligomer, and at least one photoinitiator. The cosmetic composition of the present disclosure may comprise at least one reactive monomer, at least one reactive oligomer and at least one photoinitiator, and the hydrophobic fumed silica may have the INCI name Silica Dimethicone Silylate, Silica Dimethyl Silylate, Silica Silylate, Silica Cetyl Silylate or Silica Capryl Silylate.

The at least one reactive monomer may be selected from the group of hydroxypropyl methacrylate (HPMA), hydroxyethyl methacrylate (HEMA), ethyl methacrylate (EMA), hydroxypropyl acrylate (HPA), tetrahydrofurfuryl methacrylate (THFMA), pyromellitic dianhydride di(meth)acrylate, pyromellitic dianhydride glyceryl dimethacrylate, pyromellitic dimethacrylate, methacroyloxyethyl maleate, 2-hydroxyethyl methacrylate/succinate, 1,3-glycerol dimethacrylate/succinate adduct, phthalic acid monoethyl methacrylate, methyl(meth)acrylate, ethyl(meth)acrylate, n-propyl(meth)acrylate, isopropyl(meth)acrylate, n-butyl(meth)acrylate, isobutyl(meth)acrylate, t-butyl(meth)acrylate, 2-ethylhexyl(meth)acrylate, cyclohexyl(meth)acrylate, stearyl(meth)acrylate, lauryl(meth)acrylate, trimethylcyclohexyl(meth)acrylate, isobornyl(meth)acrylate, or other alkyl(meth)acrylates; phenyl(meth)acrylate, benzyl(meth)acrylate, phenoxyethyl(meth)acrylate, phenoxydiethylene glycol (meth)acrylate, or other aromatic (meth)acrylates; oxetane (meth)acrylate, or other heterocyclic (meth)acrylates; methoxy polypropylene glycol (meth)acrylate, ethoxy polypropylene glycol (meth)acrylate, or other alkoxy polyalkylene glycol (meth)acrylates; neopentyl glycol (meth)acrylate, Triethylene Glycol Dimethacrylate, polypropylene glycol esters of (meth)acrylic acid, Trifluoroethyl Methacrylate, Trimethylolpropane Trimethacrylate, PPG-5 methacrylate (Polypropylene glycol-5 methacrylate), Di-HEMA trimethylhexyl dicarbamate, (meth)acrylamide, N,N-dimethyl(meth)acrylamide, N,N-diethyl(meth)acrylamide, N-isopropyl (meth)acrylamide, diacetone (meth)acrylamide, acryloylmorpholine, or other N-substituted (meth)acrylamides; N,N-dimethylaminoethyl(meth)acrylate, N,N-diethylaminoethyl(meth)acrylate, or other carboxyl groups containing (meth)acrylates; or (meth)acrylonitrile, or other nitriles, and mixtures thereof. In this disclosure, the term (meth)acrylate encompasses acrylates and/or methacrylates.

The at least one reactive oligomer may be selected from the group of Bis-Aminopropyl Dimethicone/IPDI Copolymer, Bis-Benzyl Butylene Glycol/Dilinoleic Acid/IPDI Copolymer, Bis-Benzyl Butylene Glycol/IPDI Copolymer, Bis-Benzyl Propanediol/IPDI Copolymer, Bis-Dicaprolactone Ethoxyacrylate Diglycol/IPDI Copolymer, Bis-Dicaprolactone Ethoxyacrylate IPDI, Bis-Ethylhexyl Poly(1,4-Butanediol)-13/IPDI Copolymer, Bis-Ethylhexyl Poly(1,4-Butanediol)-14/IPDI Copolymer, Bis-Ethylhexyl Poly(1,4-Butanediol)-22/IPDI Copolymer, Bis-Ethylhexyl Poly(1,4-Butanediol)-22/DMPA/Neopentyl Glycol/IPDI Copolymer, Bis-Ethylhexyl Poly(Caprolactone Neopentyl Glycol)/IPDI Copolymer, Bis-Ethylhexyl Poly(Caprolactone/Pentaerythritol)/IPDI Copolymer, Bis-Ethylhexyl Poly(Caprolactone/Pentaerythritol)/IPDI Copolymer Succinate, Bis-Glyceryl Dimethacrylate Poly(1,4-Butanediol)-18/IPDI Copolymer, Bis-HEA 1,4-Butanediol/IPDI Copolymer, Bis-HEA/Ethylhexyl Poly(1,4-Butanediol)-11/IPDI Copolymer, Bis-HEA/HEMA Poly(1,4-Butanediol)-41/1,2-Butanediol/IPDI Copolymer, Bis-HEA/HPMA PEG-6/Poly(1,4-Butanediol)-28/IPDI Copolymer, Bis-HEA IPDI/PPG-26 Copolymer, Bis-HEA IPDI/PPG-53 Copolymer, Bis-HEA Isopropylidenediphenyl Bisoxyhydroxypropyl Ether/IPDI Copolymer, Bis-HEA Pentaerythrityl Triacrylate Poly(1,4-Butanediol)-9/IPDI Copolymer, Bis-HEA Poly(1,4-Butanediol)-9/IPDI Copolymer, Bis-HEA Poly(1,4-Butanediol)-22/IPDI Copolymer, Bis-HEA Poly(Caprolactone Diglycol)/IPDI Copolymer, Bis-HEA Polycaprolactone Diol/IPDI Copolymer, Bis-(HEA Polycaprolactone) PEG-2/PPG-3/IPDI Copolymer, Bis-HEA Polydiethyleneglycol Adipate/IPDI Copolymer, Bis-HEA Polyglycol Adipate/IPDI Copolymer, Bis-HEA Poly(Glycol/1,4-Butanediol Adipate)/IPDI Copolymer, Bis-HEA Poly(Hexamethylene/Methylpentylene Carbonate)/IPDI Copolymer, Bis-HEA Tricyclodecanedimethyl Polycaprolactone Diol/IPDI Copolymer, Bis-(HEMA/Ethylhexyl) Poly(1,4-Butanediol)-9/TMDI/IPDI Copolymer, Bis-HEMA IPDI, Bis-HEMA IPDI/PEG-4 Copolymer, Bis-HEMA IPDI/PPG-12 Copolymer, Bis-HEMA Poloxamer 251/IPDI Copolymer, Bis-HEMA Poly(1,4-Butanediol Adipate)/IPDI Copolymer, Bis-HEMA Poly(1,4-Butanediol)-13/1,4-Butanediol/IPDI Copolymer, Bis-HEMA Poly(1,4-Butanediol)-22/DMPA/Neopentyl Glycol/IPDI Copolymer, Bis-HEMA Poly(1,4-Butanediol)-9/IPDI Copolymer, Bis-HEMA Poly(1,4-Butanediol)-14/IPDI Copolymer, Bis-HEMA Poly(1,4-Butanediol)-22/IPDI Copolymer, Bis-HEMA Poly(1,4-Butanediol)-14 Neopentyl Glycol/IPDI Copolymer, Bis-HEMA Poly(1,4-Butanediol)-14/PEG-2/PPG-3/IPDI Copolymer, Bis-HEMA Poly(Butylene/Hexamethylene Carbonate)/IPDI Copolymer, Bis-HEMA Polycaprolactone Diol/IPDI Copolymer, Bis-HEMA Poly(Caprolactone Hexanediol)/IPDI Copolymer, BIS-HEMA Polycaprolactone/IPDI Copolymer, Bis-HEMA Poly(Caprolactone Neopentyl Glycol)/1,4 Butanediol/IPDI Copolymer, Bis-HEMA Poly(Caprolactone Neopentyl Glycol)/IPDI Copolymer, Bis-HEMA Poly(Caprolactone/Pentaerythritol)/IPDI Copolymer, Bis-HEMA Poly(Caprolactone/Pentaerythritol)/IPDI Copolymer Succinate, Bis-HEMA Polycaprolactone/Poly(1,4-Butanediol)-6/IPDI Copolymer, Bis-HEMA Poly(Diglycol Adipate)/IPDI Copolymer, Bis-HEMA Poly(Diglycol/Glycol Adipate)/IPDI Copolymer, Bis-HEMA Poly(Dimethylcyclohexane/Hexane Carbonate)/IPDI Copolymer, Bis-HEMA Poly(Glycol/TMP Adipate)/IPDI Copolymer, Bis-HEMA Poly(Hexamethylene/Ethylene Carbonate)/IPDI Copolymer, Bis-HEMA Poly(Methylpentanediol Adipate)/IPDI Copolymer, Bis-HEMA Polyneopentyl Glycol Adipate/IPDI Copolymer, Bis-HEMA Poly(Pentylene/Hexamethylene Carbonate)/IPDI Copolymer, Bis-HEMA Poly(Propylene Glycol Adipate)/IPDI Copolymer, Bis-HEMA PPG-17/IPDI Copolymer, Bis-HEMA PPG-26/IPDI Copolymer, Bis-HPA Poly(1,4-Butanediol)-9/Glycol/IPDI Copolymer, Bis-Hydroxyethoxypropyl Dimethicone/IPDI Copolymer Ethylcarbamate, Bis-Hydroxyethoxypropyl Dimethicone/IPDI/Sorbitan Stearate Copolymer, Bis-Hydroxyethoxypropyl Dimethicone/PEG-2 Soyamine/IPDI Copolymer, Bis-Hydroxyethyl Acrylate Poly(1,4-Butanediol)-9/IPDI Copolymer, Bis-Hydroxyethylacrylate Polyhexamethylene Carbonate/IPDI Copolymer, Bis-Hydroxyethyl Acrylate Poly(Neopentyl Glycol Adipate)/IPDI Copolymer, Bis-Hydroxylauryl Dimethicone/IPDI Copolymer, Bis-Hydroxypropylmethacrylate Poly(1,4-Butanediol)-9/IPDI Copolymer, Bis-Hydroxypropyltrisiloxyl IPDI/PEG-14 Copolymer, Bis-Hydroxypropyltrisiloxyl IPDI/PEG-23 Copolymer, Bis-Lauryl Lactate IPDI/PEG-2 Soyamine Copolymer, Bis-Methoxy PEG-114 Polycaprolactone Diol/IPDI Copolymer, Bis-Methylisocytosine Hydrogenated Polybutadiene/IPDI Copolymer, Bis-PEG-15 Dimethicone/IPDI Copolymer, Bis-PEG-1 Dimethicone/PPG-26/IPDI Copolymer, Bis-Pentaerythrityl Triacrylate Glycol/Poly(1,4-Butanediol)-9/IPDI Copolymer, Bis(Pentaerythrityl Triacrylate) Pentaerythrityl Diacrylate/IPDI Copolymer, Bis-Pentaerythrityl Triacrylate Poly(1,4-Butanediol)-9/Glycol/IPDI Copolymer, Bis-PPG-15 Dimethicone/IPDI Copolymer, Bis-PPG-15 Dimethicone IPDI Copolymer C30-50 Alkylcarbamate, Bis-Stearyl IPDI/PEG-795 Copolymer, Bis-(TMP Diallyl Ether) IPDI, Dicaprolactone Ethoxyacrylate/1,4-Butanediol/IPDI Copolymer, Dicaprolactone Ethoxyacrylate HEMA IPDI, Isopropylidenediphenol PEG-2 Dimethacrylate, Isopropylidenediphenol PEG-4 Diacrylate, Isopropylidenediphenyl Bisoxyhydroxypropyl Methacrylate/TMDI Copolymer, Isopropylidenediphenyl Bisoxyhydroxypropyl Methacrylate, Isopropylidenediphenyl Bisoxyhydroxypropyl Acrylate, and mixtures thereof. In this disclosure, the term “IPDI” means Isophorone Diisocyanate; “DMPA” means Dimethylaminopropylamine; “TMP” means Trimethylolpropane; “TMDI” means Trimethylhexamethylene Diisocyanate; “PPG” means Polypropylene glycol; and “PEG” means Polyethylene glycol.

The at least one photoinitiator may be selected from the group of 1-hydroxycyclohexyl phenyl ketone, benzyl dimethyl ketal, phenyldimethoxyacetophenone, ethyl trimethyl 2-hydroxy-2-methyl-1-phenyl propan-1-one, bis(2,6-dimethoxybenzoyl) 2,4,4-trimethylpentyl phosphine oxide (DMBAPO), bis(2,4,6-trimethylbenzoyl) phenyl phosphineoxide, 2,4,6-trimethylbenzyldiphenyl phosphine oxide, ethyl trimethylbenzoyl phenylphosphinate, 2,4,6-trimethylbenzoyl diphenylphosphine oxide, trimethylbenzoyl diphenylphosphine oxide, bis-trimethylbenzoyl phenylphosphine oxide, benzoin methyl ester, camphorquinone, ethyl 4-(N,N-dimethylamino)benzoate, and mixtures thereof.

The at least one reactive monomer may be present in an amount between 10% and 40%, between 25% and 37%, between 28% and 35% by weight of the total weight of the cosmetic composition. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive monomer may be present in an amount between 10% and 40%, between 25% and 37%, between 28% and 35% by weight of the total weight of the cosmetic composition. The sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive monomer may be present in an amount between 10% and 40%, between 25% and 37%, between 28% and 35% by weight of the total weight of the cosmetic composition. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive monomer may be present in an amount between 10% and 40%, between 25% and 37%, between 28% and 35% by weight of the total weight of the cosmetic composition.

The at least one reactive monomer may be present in an amount between 10% and 40%, between 25% and 37%, between 28% and 35% by weight of the total weight of the cosmetic composition. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive monomer may be present in an amount between 10% and 40%, between 25% and 37%, between 28% and 35% by weight of the total weight of the cosmetic composition. The sum of the at least one hydrophobic fumed silica and the at least one hydrophilic fumed silica may range from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive monomer may be present in an amount between 10% and 40%, between 25% and 37%, between 28% and 35% by weight of the total weight of the cosmetic composition. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 1.75% and 3.0% by weight of the total weight of the cosmetic composition, the sum of the at least one hydrophobic fumed silica and the at least one hydrophilic fumed silica may range from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive monomer may be present in an amount between 10% and 40%, between 25% and 37%, between 28% and 35% by weight of the total weight of the cosmetic composition.

The at least one reactive monomer may be present in an amount between 10% and 40%, between 25% and 37%, between 28% and 35% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive monomer may be present in an amount between 10% and 40%, between 25% and 37%, between 28% and 35% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive monomer may be present in an amount between 10% and 40%, between 25% and 37%, between 28% and 35% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive monomer may be present in an amount between 10% and 40%, between 25% and 37%, between 28% and 35% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate.

The at least one reactive monomer may be present in an amount between 10% and 40%, between 25% and 37%, between 28% and 35% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive monomer may be present in an amount between 10% and 40%, between 25% and 37%, between 28% and 35% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The sum of the at least one hydrophobic fumed silica and the at least one hydrophilic fumed silica may range from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive monomer may be present in an amount between 10% and 40%, between 25% and 37%, between 28% and 35% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 1.75% and 3.0% by weight of the total weight of the cosmetic composition, the sum of the at least one hydrophobic fumed silica and the at least one hydrophilic fumed silica may range from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive monomer may be present in an amount between 10% and 40%, between 25% and 37%, between 28% and 35% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate.

The at least one reactive oligomer may be present in an amount between 40% and 60%, between 42% and 53%, between 45% and 49% by weight of the total weight of the cosmetic composition. The at least one reactive monomer may be present in an amount between 10% and 40%, between 25% and 37%, between 28% and 35% by weight of the total weight of the cosmetic composition, and the at least one reactive oligomer may be present in an amount between 40% and 60%, between 42% and 53%, between 45% and 49% by weight of the total weight of the cosmetic composition. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive oligomer may be present in an amount between 40% and 60%, between 42% and 53%, between 45% and 49% by weight of the total weight of the cosmetic composition. The at least one reactive monomer may be present in an amount between 10% and 40%, between 25% and 37%, between 28% and 35% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive oligomer may be present in an amount between 40% and 60%, between 42% and 53%, between 45% and 49% by weight of the total weight of the cosmetic composition. The sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive oligomer may be present in an amount between 40% and 60%, between 42% and 53%, between 45% and 49% by weight of the total weight of the cosmetic composition. The at least one reactive monomer may be present in an amount between 10% and 40%, between 25% and 37%, between 28% and 35% by weight of the total weight of the cosmetic composition, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive oligomer may be present in an amount between 40% and 60%, between 42% and 53%, between 45% and 49% by weight of the total weight of the cosmetic composition. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive oligomer may be present in an amount between 40% and 60%, between 42% and 53%, between 45% and 49% by weight of the total weight of the cosmetic composition. The at least one reactive monomer may be present in an amount between 10% and 40%, between 25% and 37%, between 28% and 35% by weight of the total weight of the cosmetic composition, the at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive oligomer may be present in an amount between 40% and 60%, between 42% and 53%, between 45% and 49% by weight of the total weight of the cosmetic composition.

The at least one reactive oligomer may be present in an amount between 40% and 60%, between 42% and 53%, between 45% and 49% by weight of the total weight of the cosmetic composition. The at least one reactive monomer may be present in an amount between 10% and 40%, between 25% and 37%, between 28% and 35% by weight of the total weight of the cosmetic composition, and the at least one reactive oligomer may be present in an amount between 40% and 60%, between 42% and 53%, between 45% and 49% by weight of the total weight of the cosmetic composition. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive oligomer may be present in an amount between 40% and 60%, between 42% and 53%, between 45% and 49% by weight of the total weight of the cosmetic composition. The at least one reactive monomer may be present in an amount between 10% and 40%, between 25% and 37%, between 28% and 35% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive oligomer may be present in an amount between 40% and 60%, between 42% and 53%, between 45% and 49% by weight of the total weight of the cosmetic composition. The sum of the at least one hydrophobic fumed silica and the at least one hydrophilic fumed silica may range from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive oligomer may be present in an amount between 40% and 60%, between 42% and 53%, between 45% and 49% by weight of the total weight of the cosmetic composition. The at least one reactive monomer may be present in an amount between 10% and 40%, between 25% and 37%, between 28% and 35% by weight of the total weight of the cosmetic composition, the sum of the at least one hydrophobic fumed silica and the at least one hydrophilic fumed silica may range from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive oligomer may be present in an amount between 40% and 60%, between 42% and 53%, between 45% and 49% by weight of the total weight of the cosmetic composition. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 1.75% and 3.0% by weight of the total weight of the cosmetic composition, the sum of the at least one hydrophobic fumed silica and the at least one hydrophilic fumed silica may range from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive oligomer may be present in an amount between 40% and 60%, between 42% and 53%, between 45% and 49% by weight of the total weight of the cosmetic composition. The at least one reactive monomer may be present in an amount between 10% and 40%, between 25% and 37%, between 28% and 35% by weight of the total weight of the cosmetic composition, the at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 1.75% and 3.0% by weight of the total weight of the cosmetic composition, the sum of the at least one hydrophobic fumed silica and the at least one hydrophilic fumed silica may range from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive oligomer may be present in an amount between 40% and 60%, between 42% and 53%, between 45% and 49% by weight of the total weight of the cosmetic composition.

The at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one reactive monomer may be present in an amount between 10% and 40%, between 25% and 37%, between 28% and 35% by weight of the total weight of the cosmetic composition, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one reactive monomer may be present in an amount between 10% and 40%, between 25% and 37%, between 28% and 35% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one reactive monomer may be present in an amount between 10% and 40%, between 25% and 37%, between 28% and 35% by weight of the total weight of the cosmetic composition, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one reactive monomer may be present in an amount between 10% and 40%, between 25% and 37%, between 28% and 35% by weight of the total weight of the cosmetic composition, the at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate.

The at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one reactive monomer may be present in an amount between 10% and 40%, between 25% and 37%, between 28% and 35% by weight of the total weight of the cosmetic composition, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one reactive monomer may be present in an amount between 10% and 40%, between 25% and 37%, between 28% and 35% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The sum of the at least one hydrophobic fumed silica and the at least one hydrophilic fumed silica may range from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one reactive monomer may be present in an amount between 10% and 40%, between 25% and 37%, between 28% and 35% by weight of the total weight of the cosmetic composition, the sum of the at least one hydrophobic fumed silica and the at least one hydrophilic fumed silica may range from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 1.75% and 3.0% by weight of the total weight of the cosmetic composition, the sum of the at least one hydrophobic fumed silica and the at least one hydrophilic fumed silica may range from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one reactive monomer may be present in an amount between 10% and 40%, between 25% and 37%, between 28% and 35% by weight of the total weight of the cosmetic composition, the at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 1.75% and 3.0% by weight of the total weight of the cosmetic composition, the sum of the at least one hydrophobic fumed silica and the at least one hydrophilic fumed silica may range from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate.

The at least one photoinitiator may be present in an amount between 0.001% and 5%, between 0.01% and 2.5% by weight of the total weight of the cosmetic composition. The photoinitiator may be used in the cosmetic composition in effective amounts to initiate photopolymerization of the reaction mixture of the at least one reactive monomer and the at least one reactive oligomer.

The cosmetic composition that may comprise at least one reactive monomer, at least one reactive oligomer, and at least one photoinitiator may optionally further comprise at least one additional cosmetic ingredient selected from the group of film formers, colorants, pigments, plasticizers and light stabilizers.

The film former may be selected from the group of nitrocellulose, ethyl cellulose, polyethyl cellulose, polyhydroxypropyl cellulose, cellulose esters, cellulose acetate alkylates such as cellulose acetate butyrate, cellulose acetate propionate; ethyl tosylamide, adipic acid/fumaric acid/phthalic acid/tricyclodecane dimethanol copolymer, adipic acid/neopentyl glycol/trimellitic anhydride copolymer, phthalic anhydride/trimellitic anhydride/glycols copolymer, polyethyl acrylate oxide, polylactic acid, polyvinyl acetate, polyvinyl chloride, polyvinylbutyrate, (meth)acrylic and vinyl copolymers such as styrene/butadiene copolymers, acrylate/vinyl acetate copolymers, acrylonitrile/butadiene copolymers, and ethylene/vinyl acetate copolymers; and mixtures thereof. The film former may be dispersed within the 3D network formed by the polymerization of the at least one reactive monomer and the at least one reactive oligomer and, when present, may help to produce a continuous film on the nail.

The film former may not be present or may be present in an amount of from more than 0% to 25%, between 8% and 20%, between 10% and 15% by weight of the total weight of the cosmetic composition.

The colorant or pigment may be selected from the group of Ext. D&C Violet No. 2, Sudan Red, DC Red 17, DC Green 6, Beta-Carotene, soybean oil, Sudan Brown, DC Yellow 11, DC Violet 2, DC Orange 5, annatto, and quinoline yellow; bismuth oxychloride, optionally surface-treated titanium dioxide, zirconium oxide, zinc oxide, cerium oxide, iron oxides, chromium oxides, manganese violet, ultramarine blue, chromium hydrate, and ferric blue,

The colorant or pigment may not be present or may be present in an amount from more than 0% to 2%, from more than 0% to 1%, from more than 0% to 0.1% by weight of the total weight of the cosmetic composition.

The plasticizer may be selected from the group of triethyl citrate (TEC), acetyl triethyl citrate (ATEC), tributyl citrate (TBC), acetyl tributyl citrate (ATBC), triamyl citrate, trioctyl citrate (TOC), acetyl trioctyl citrate (ATOC), trihexyl citrate (THC), acetyl trihexyl citrate (ATHC), butyroyl trihexyl citrate (BTHC, or trihexyl butyroyl citrate), trimethyl citrate (TMC), alkyl sulphonic acid phenyl ester (ASE), acetylated monoglyceride, dibutyl phthalate, dioctyl phthalate, tricresyl phthalate, butyl phthalate, dibutoxy ethyl phthalate, diamyl phthalate, tosyl amide, N-ethyl-tosyl amide, sucrose acetate isobutyrate, trimethyl pentanyl diisobutyrate, camphor, castor oil, glyceryl diesters, glyceryl triesters, tributyl phosphate, triphenyl phosphate, butyl glycolate, benzyl benzoate, butyl acetyl ricinoleate, butyl stearate, dibutyl tartrate, diethylene glycol ethyl ether, diethylene glycol methyl ether, diethylene glycol butyl ether, diethylene glycol hexyl ether, ethylene glycol ethyl ether, ethylene glycol butyl ether and ethylene glycol hexyl ether, propylene glycol derivatives including propylene glycol phenyl ether, propylene glycol diacetate, dipropylene glycol butyl ether, tripropylene glycol butyl ether, propylene glycol methyl ether, dipropylene glycol ethyl ether, tripropylene glycol methyl ether, propylene glycol butyl ether, isostearyl isononanoate, diisobutyl adipate, vinyl chloride copolymer, 1,2-cyclohexane dicarboxylic acid diisononyl ester, and mixtures thereof. The plasticizer may help improve flexibility and/or reduce brittleness of the polymerized gel sculpted nail composition.

The plasticizer may not be present or may be present in an amount from more than 0% to 10%, from more than 0% to 5%, from more than 0% to 2% by weight of the total weight of the cosmetic composition.

The light stabilizer may be selected from the group of hydroxy benzotriazole compounds such as 2-(2-hydroxy-5′-methylphenyl)benzotriazole, benzophenones 1-12, 3-benzylidene camphor, benzyl salicylate, borneolone, borneol, camphor, bumetrizole, PABA (para-aminobenzoic acid), butyl PABA, butyl methoxydibenzoylmethane, cinoxate, DEA-methoxycinnamate (diethanolamine-methoxycinnamate), dibenzoxazoyl naphthalene, digalloyl trioleate, diisopropyl methyl cinnamate, BHT (butylated hydroxytoluene) and mixtures thereof. The light stabilizer may help improve the shelf-life of the composition and/or reduce the yellowish color, which is often seen in artificial nails.

The light stabilizer may not be present or may be present in an amount from more than 0% to 2%, from more than 0% to 1%, from more than 0% to 0.25% by weight of the total weight of the cosmetic composition.

The present disclosure also relates to a method of sculpting an artificial nail onto at least a portion of a nail, the method comprising: applying onto at least a portion of the nail a layer of a cosmetic composition which comprises at least one hydrophobic fumed silica and at least one hydrophilic fumed silica, wherein the cosmetic composition is a gel; sculpting the cosmetic composition into a desired shape and length; and curing the cosmetic composition.

The method of sculpting an artificial nail of the present disclosure includes the formation of an artificial nail of a desired shape and length upon a portion of or the whole nail surface, and optionally upon a plastic, paper or cardboard fingernail extension form joined to the finger.

In the present disclosure, “cure” or “curing” means the polymerization of a nail cosmetic composition by means of radiation, such as from a nail lamp.

In the method of the present disclosure, the applying, sculpting, and curing of the cosmetic composition may be repeated at least once, at least twice per nail.

In the method of the present disclosure, the nail lamp for curing of the cosmetic composition may be a visible light or UV nail lamp.

The curing of the cosmetic composition in the method of the present disclosure may last for at least 10 seconds, at least 30 seconds, at least 60 seconds, at least 2 minutes.

The layer of the cosmetic composition in the method of the present disclosure may have a thickness of at least 1 mm, at least 1.5 mm.

The present disclosure also relates to a brush-in bottle, wherein the bottle contains a cosmetic composition comprising at least one hydrophobic fumed silica and at least one hydrophilic fumed silica, wherein the cosmetic composition is a gel sculpting nail composition applied or applicable to at least a portion of a nail with the brush.

In the present disclosure, the term “brush-in bottle” means a bottle wherein a brush is inside of the bottle and joined to the cap of the bottle. When the brush-in bottle is fully or partially closed, the brush may be submerged within the cosmetic composition contained in the bottle.

The cosmetic composition in the brush-in bottle has a viscosity thin enough to be packaged like a gel polish, such as in a bottle. The gel sculpting nail composition has a viscosity thin enough to be applied to the nail like a gel polish, i.e. by painting, without sagging, and with a thickness of the layers that allows a low number of layers to be applied, and therefore, saves time in the process of sculpting nails.

In the cosmetic composition within the brush-in bottle of the present disclosure, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1. In the cosmetic composition within the brush-in bottle of the present disclosure, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the hydrophobic fumed silica may have the INCI name Silica Dimethicone Silylate, Silica Dimethyl Silylate, Silica Silylate, Silica Cetyl Silylate or Silica Capryl Silylate.

In the cosmetic composition within the brush-in bottle of the present disclosure, the at least one hydrophobic fumed silica may be present in an amount between 0.1% and 6%, between 0.2% and 4.0%, between 0.3% and 2.5%, between 0.4% and 2.0% by weight of the total weight of the cosmetic composition. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one hydrophobic fumed silica may be present in an amount between 0.1% and 6%, between 0.2% and 4.0%, between 0.3% and 2.5%, between 0.4% and 2.0% by weight of the total weight of the cosmetic composition.

In the cosmetic composition within the brush-in bottle of the present disclosure, the at least one hydrophobic fumed silica may be present in an amount between 0.1% and 6%, between 0.2% and 4.0%, between 0.3% and 2.5%, between 0.4% and 2.0% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one hydrophobic fumed silica may be present in an amount between 0.1% and 6%, between 0.2% and 4.0%, between 0.3% and 2.5%, between 0.4% and 2.0% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate.

In the cosmetic composition within the brush-in bottle of the present disclosure, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, and the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one hydrophobic fumed silica may be present in an amount between 0.1% and 6%, between 0.2% and 4.0%, between 0.3% and 2.5%, between 0.4% and 2.0% by weight of the total weight of the cosmetic composition, and the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition.

In the cosmetic composition within the brush-in bottle of the present disclosure, the at least one hydrophilic fumed silica may be present in an amount between 1.75% and 3.0% by weight of the total weight of the cosmetic composition. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one hydrophilic fumed silica may be present in an amount between 1.75% and 3.0% by weight of the total weight of the cosmetic composition. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, and the at least one hydrophilic fumed silica may be present in an amount between 1.75% and 3.0% by weight of the total weight of the cosmetic composition. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one hydrophobic fumed silica may be present in an amount between 0.1% and 6%, between 0.2% and 4.0%, between 0.3% and 2.5%, between 0.4% and 2.0% by weight of the total weight of the cosmetic composition, and the at least one hydrophilic fumed silica may be present in an amount between 1.75% and 3.0% by weight of the total weight of the cosmetic composition.

In the cosmetic composition within the brush-in bottle of the present disclosure, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate.

In the cosmetic composition within the brush-in bottle of the present disclosure, the at least one hydrophilic fumed silica may be present in an amount between 1.75% and 3.0% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one hydrophilic fumed silica may be present in an amount between 1.75% and 3.0% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 1.75% and 3.0% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 1.75% and 3.0% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate.

In the cosmetic composition within the brush-in bottle of the present disclosure, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, and the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition. The at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, and the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, and the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, and the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition.

In the cosmetic composition within the brush-in bottle of the present disclosure, the sum of the at least one hydrophobic fumed silica and the at least one hydrophilic fumed silica may range from 2.2% to 4.2% by weight of the total weight of the cosmetic composition. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, and the sum of the at least one hydrophobic fumed silica and the at least one hydrophilic fumed silica may range from 2.2% to 4.2% by weight of the total weight of the cosmetic composition. The at least one hydrophilic fumed silica may be present in an amount between 1.75% and 3.0% by weight of the total weight of the cosmetic composition, and the sum of the at least one hydrophobic fumed silica and the at least one hydrophilic fumed silica may range from 2.2% to 4.2% by weight of the total weight of the cosmetic composition. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the sum of the at least one hydrophobic fumed silica and the at least one hydrophilic fumed silica may range from 2.2% to 4.2% by weight of the total weight of the cosmetic composition. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 1.75% and 3.0% by weight of the total weight of the cosmetic composition, and the sum of the at least one hydrophobic fumed silica and the at least one hydrophilic fumed silica may range from 2.2% to 4.2% by weight of the total weight of the cosmetic composition. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 1.75% and 3.0% by weight of the total weight of the cosmetic composition, and the sum of the at least one hydrophobic fumed silica and the at least one hydrophilic fumed silica may range from 2.2% to 4.2% by weight of the total weight of the cosmetic composition.

In the cosmetic composition within the brush-in bottle of the present disclosure, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate.

In the cosmetic composition within the brush-in bottle of the present disclosure, the sum of the at least one hydrophobic fumed silica and the at least one hydrophilic fumed silica may range from 2.2% to 4.2% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the sum of the at least one hydrophobic fumed silica and the at least one hydrophilic fumed silica may range from 2.2% to 4.2% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one hydrophilic fumed silica may be present in an amount between 1.75% and 3.0% by weight of the total weight of the cosmetic composition, the sum of the at least one hydrophobic fumed silica and the at least one hydrophilic fumed silica may range from 2.2% to 4.2% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the sum of the at least one hydrophobic fumed silica and the at least one hydrophilic fumed silica may range from 2.2% to 4.2% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 1.75% and 3.0% by weight of the total weight of the cosmetic composition, the sum of the at least one hydrophobic fumed silica and the at least one hydrophilic fumed silica may range from 2.2% to 4.2% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 1.75% and 3.0% by weight of the total weight of the cosmetic composition, the sum of the at least one hydrophobic fumed silica and the at least one hydrophilic fumed silica may range from 2.2% to 4.2% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate.

The cosmetic composition within the brush-in bottle of the present disclosure may comprise at least one reactive monomer, at least one reactive oligomer, and at least one photoinitiator.

In the cosmetic composition within the brush-in bottle of the present disclosure, the at least one reactive monomer may be selected from the group of hydroxypropyl methacrylate (HPMA), hydroxyethyl methacrylate (HEMA), ethyl methacrylate (EMA), hydroxypropyl acrylate (HPA), tetrahydrofurfuryl methacrylate (THFMA), pyromellitic dianhydride di(meth)acrylate, pyromellitic dianhydride glyceryl dimethacrylate, pyromellitic dimethacrylate, methacroyloxyethyl maleate, 2-hydroxyethyl methacrylate/succinate, 1,3-glycerol dimethacrylate/succinate adduct, phthalic acid monoethyl methacrylate, methyl(meth)acrylate, ethyl(meth)acrylate, n-propyl(meth)acrylate, isopropyl(meth)acrylate, n-butyl(meth)acrylate, isobutyl(meth)acrylate, t-butyl(meth)acrylate, 2-ethylhexyl(meth)acrylate, cyclohexyl(meth)acrylate, stearyl(meth)acrylate, lauryl(meth)acrylate, trimethyl cyclohexyl(meth)acrylate, isobornyl(meth)acrylate, or other alkyl(meth)acrylates; phenyl(meth)acrylate, benzyl(meth)acrylate, phenoxyethyl(meth)acrylate, phenoxydiethylene glycol (meth)acrylate, or other aromatic (meth)acrylates; oxetane (meth)acrylate, or other heterocyclic (meth)acrylates; methoxy polypropylene glycol (meth)acrylate, ethoxy polypropylene glycol (meth)acrylate, or other alkoxy polyalkylene glycol (meth)acrylates; neopentyl glycol (meth)acrylate, Triethylene Glycol Dimethacrylate, polypropylene glycol esters of (meth)acrylic acid, Trifluoroethyl Methacrylate, Trimethylolpropane Trimethacrylate, PPG-5 methacrylate, Di-HEMA trimethylhexyl dicarbamate, (meth)acrylamide, N,N-dimethyl(meth)acrylamide, N,N-di ethyl(meth)acrylamide, N-isopropyl(meth)acrylamide, diacetone (meth)acrylamide, acryloylmorpholine, or other N-substituted (meth)acrylamides; N,N-dimethylaminoethyl(meth)acrylate, N,N-diethylaminoethyl(meth)acrylate, or other carboxyl groups containing (meth)acrylates; or (meth)acrylonitrile, or other nitriles, and mixtures thereof.

In the cosmetic composition within the brush-in bottle of the present disclosure, the at least one oligomer may be selected from the group of Bis-Aminopropyl Dimethicone/IPDI Copolymer, Bis-Benzyl Butylene Glycol/Dilinoleic Acid/IPDI Copolymer, Bis-Benzyl Butylene Glycol/IPDI Copolymer, Bis-Benzyl Propanediol/IPDI Copolymer, Bis-Dicaprolactone Ethoxyacrylate Diglycol/IPDI Copolymer, Bis-Dicaprolactone Ethoxyacrylate IPDI, Bis-Ethylhexyl Poly(1,4-Butanediol)-13/IPDI Copolymer, Bis-Ethylhexyl Poly(1,4-Butanediol)-14/IPDI Copolymer, Bis-Ethylhexyl Poly(1,4-Butanediol)-22/IPDI Copolymer, Bis-Ethylhexyl Poly(1,4-Butanediol)-22/DMPA/Neopentyl Glycol/IPDI Copolymer, Bis-Ethylhexyl Poly(Caprolactone Neopentyl Glycol)/IPDI Copolymer, Bis-Ethylhexyl Poly(Caprolactone/Pentaerythritol)/IPDI Copolymer, Bis-Ethylhexyl Poly(Caprolactone/Pentaerythritol)/IPDI Copolymer Succinate, Bis-Glyceryl Dimethacrylate Poly(1,4-Butanediol)-18/IPDI Copolymer, Bis-HEA 1,4-Butanediol/IPDI Copolymer, Bis-HEA/Ethylhexyl Poly(1,4-Butanediol)-11/IPDI Copolymer, Bis-HEA/HEMA Poly(1,4-Butanediol)-41/1,2-Butanediol/IPDI Copolymer, Bis-HEA/HPMA PEG-6/Poly(1,4-Butanediol)-28/IPDI Copolymer, Bis-HEA IPDI/PPG-26 Copolymer, Bis-HEA IPDI/PPG-53 Copolymer, Bis-HEA Isopropylidenediphenyl Bisoxyhydroxypropyl Ether/IPDI Copolymer, Bis-HEA Pentaerythrityl Triacrylate Poly(1,4-Butanediol)-9/IPDI Copolymer, Bis-HEA Poly(1,4-Butanediol)-9/IPDI Copolymer, Bis-HEA Poly(1,4-Butanediol)-22/IPDI Copolymer, Bis-HEA Poly(Caprolactone Diglycol)/IPDI Copolymer, Bis-HEA Polycaprolactone Diol/IPDI Copolymer, Bis-(HEA Polycaprolactone) PEG-2/PPG-3/IPDI Copolymer, Bis-HEA Polydiethyleneglycol Adipate/IPDI Copolymer, Bis-HEA Polyglycol Adipate/IPDI Copolymer, Bis-HEA Poly(Glycol/1,4-Butanediol Adipate)/IPDI Copolymer, Bis-HEA Poly(Hexamethylene/Methylpentylene Carbonate)/IPDI Copolymer, Bis-HEA Tricyclodecanedimethyl Polycaprolactone Diol/IPDI Copolymer, Bis-(HEMA/Ethylhexyl) Poly(1,4-Butanediol)-9/TMDI/IPDI Copolymer, Bis-HEMA IPDI, Bis-HEMA IPDI/PEG-4 Copolymer, Bis-HEMA IPDI/PPG-12 Copolymer, Bis-HEMA Poloxamer 251/IPDI Copolymer, Bis-HEMA Poly(1,4-Butanediol Adipate)/IPDI Copolymer, Bis-HEMA Poly(1,4-Butanediol)-13/1,4-Butanediol/IPDI Copolymer, Bis-HEMA Poly(1,4-Butanediol)-22/DMPA/Neopentyl Glycol/IPDI Copolymer, Bis-HEMA Poly(1,4-Butanediol)-9/IPDI Copolymer, Bis-HEMA Poly(1,4-Butanediol)-14/IPDI Copolymer, Bis-HEMA Poly(1,4-Butanediol)-22/IPDI Copolymer, Bis-HEMA Poly(1,4-Butanediol)-14 Neopentyl Glycol/IPDI Copolymer, Bis-HEMA Poly(1,4-Butanediol)-14/PEG-2/PPG-3/IPDI Copolymer, Bis-HEMA Poly(Butylene/Hexamethylene Carbonate)/IPDI Copolymer, Bis-HEMA Polycaprolactone Diol/IPDI Copolymer, Bis-HEMA Poly(Caprolactone Hexanediol)/IPDI Copolymer, BIS-HEMA Polycaprolactone/IPDI Copolymer, Bis-HEMA Poly(Caprolactone Neopentyl Glycol)/1,4 Butanediol/IPDI Copolymer, Bis-HEMA Poly(Caprolactone Neopentyl Glycol)/IPDI Copolymer, Bis-HEMA Poly(Caprolactone/Pentaerythritol)/IPDI Copolymer, Bis-HEMA Poly(Caprolactone/Pentaerythritol)/IPDI Copolymer Succinate, Bis-HEMA Polycaprolactone/Poly(1,4-Butanediol)-6/IPDI Copolymer, Bis-HEMA Poly(Diglycol Adipate)/IPDI Copolymer, Bis-HEMA Poly(Diglycol/Glycol Adipate)/IPDI Copolymer, Bis-HEMA Poly(Dimethylcyclohexane/Hexane Carbonate)/IPDI Copolymer, Bis-HEMA Poly(Glycol/TMP Adipate)/IPDI Copolymer, Bis-HEMA Poly(Hexamethylene/Ethylene Carbonate)/IPDI Copolymer, Bis-HEMA Poly(Methylpentanediol Adipate)/IPDI Copolymer, Bis-HEMA Polyneopentyl Glycol Adipate/IPDI Copolymer, Bis-HEMA Poly(Pentylene/Hexamethylene Carbonate)/IPDI Copolymer, Bis-HEMA Poly(Propylene Glycol Adipate)/IPDI Copolymer, Bis-HEMA PPG-17/IPDI Copolymer, Bis-HEMA PPG-26/IPDI Copolymer, Bis-HPA Poly(1,4-Butanediol)-9/Glycol/IPDI Copolymer, Bis-Hydroxyethoxypropyl Dimethicone/IPDI Copolymer Ethylcarbamate, Bis-Hydroxyethoxypropyl Dimethicone/IPDI/Sorbitan Stearate Copolymer, Bis-Hydroxyethoxypropyl Dimethicone/PEG-2 Soyamine/IPDI Copolymer, Bis-Hydroxyethyl Acrylate Poly(1,4-Butanediol)-9/IPDI Copolymer, Bis-Hydroxyethylacrylate Polyhexamethylene Carbonate/IPDI Copolymer, Bis-Hydroxyethyl Acrylate Poly(Neopentyl Glycol Adipate)/IPDI Copolymer, Bis-Hydroxylauryl Dimethicone/IPDI Copolymer, Bis-Hydroxypropylmethacrylate Poly(1,4-Butanediol)-9/IPDI Copolymer, Bis-Hydroxypropyltrisiloxyl IPDI/PEG-14 Copolymer, Bis-Hydroxypropyltrisiloxyl IPDI/PEG-23 Copolymer, Bis-Lauryl Lactate IPDI/PEG-2 Soyamine Copolymer, Bis-Methoxy PEG-114 Polycaprolactone Diol/IPDI Copolymer, Bis-Methylisocytosine Hydrogenated Polybutadiene/IPDI Copolymer, Bis-PEG-15 Dimethicone/IPDI Copolymer, Bis-PEG-1 Dimethicone/PPG-26/IPDI Copolymer, Bis-Pentaerythrityl Triacrylate Glycol/Poly(1,4-Butanediol)-9/IPDI Copolymer, Bis(Pentaerythrityl Triacrylate) Pentaerythrityl Diacrylate/IPDI Copolymer, Bis-Pentaerythrityl Triacrylate Poly(1,4-Butanediol)-9/Glycol/IPDI Copolymer, Bis-PPG-15 Dimethicone/IPDI Copolymer, Bis-PPG-15 Dimethicone IPDI Copolymer C30-50 Alkylcarbamate, Bis-Stearyl IPDI/PEG-795 Copolymer, Bis-(TMP Diallyl Ether) IPDI, Dicaprolactone Ethoxyacrylate/1,4-Butanediol/IPDI Copolymer, Dicaprolactone Ethoxyacrylate HEMA IPDI, Isopropylidenediphenol PEG-2 Dimethacrylate, Isopropylidenediphenol PEG-4 Diacrylate, Isopropylidenediphenyl Bisoxyhydroxypropyl Methacrylate/TMDI Copolymer, Isopropylidenediphenyl Bisoxyhydroxypropyl Methacrylate, Isopropylidenediphenyl Bisoxyhydroxypropyl Acrylate, and mixtures thereof.

In the cosmetic composition within the brush-in bottle of the present disclosure, the at least one photoinitiator may be selected from the group of 1-hydroxycyclohexyl phenyl ketone, benzyl dimethyl ketal, phenyldimethoxyacetophenone, ethyl trimethyl 2-hydroxy-2-methyl-1-phenyl propan-1-one, bis(2,6-dimethoxybenzoyl) 2,4,4-trimethylpentyl phosphine oxide (DMBAPO), bis(2,4,6-trimethylbenzoyl) phenyl phosphineoxide, 2,4,6-trimethylbenzyldiphenyl phosphine oxide, ethyl trimethylbenzoyl phenylphosphinate, 2,4,6-trimethylbenzoyl diphenylphosphine oxide, trimethylbenzoyl diphenylphosphine oxide, bis-trimethylbenzoyl phenylphosphine oxide, benzoin methyl ester, camphorquinone, ethyl 4-(N,N-dimethylamino)benzoate, and mixtures thereof.

In the cosmetic composition within the brush-in bottle of the present disclosure, the at least one reactive monomer may be present in an amount between 10% and 40%, between 25% and 37%, between 28% and 35% by weight of the total weight of the cosmetic composition. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive monomer may be present in an amount between 10% and 40%, between 25% and 37%, between 28% and 35% by weight of the total weight of the cosmetic composition. The sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive monomer may be present in an amount between 10% and 40%, between 25% and 37%, between 28% and 35% by weight of the total weight of the cosmetic composition. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive monomer may be present in an amount between 10% and 40%, between 25% and 37%, between 28% and 35% by weight of the total weight of the cosmetic composition.

In the cosmetic composition within the brush-in bottle of the present disclosure, the at least one reactive monomer may be present in an amount between 10% and 40%, between 25% and 37%, between 28% and 35% by weight of the total weight of the cosmetic composition. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive monomer may be present in an amount between 10% and 40%, between 25% and 37%, between 28% and 35% by weight of the total weight of the cosmetic composition. The sum of the at least one hydrophobic fumed silica and the at least one hydrophilic fumed silica may range from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive monomer may be present in an amount between 10% and 40%, between 25% and 37%, between 28% and 35% by weight of the total weight of the cosmetic composition. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 1.75% and 3.0% by weight of the total weight of the cosmetic composition, the sum of the at least one hydrophobic fumed silica and the at least one hydrophilic fumed silica may range from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive monomer may be present in an amount between 10% and 40%, between 25% and 37%, between 28% and 35% by weight of the total weight of the cosmetic composition.

In the cosmetic composition within the brush-in bottle of the present disclosure, the at least one reactive monomer may be present in an amount between 10% and 40%, between 25% and 37%, between 28% and 35% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive monomer may be present in an amount between 10% and 40%, between 25% and 37%, between 28% and 35% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive monomer may be present in an amount between 10% and 40%, between 25% and 37%, between 28% and 35% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive monomer may be present in an amount between 10% and 40%, between 25% and 37%, between 28% and 35% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate.

In the cosmetic composition within the brush-in bottle of the present disclosure, the at least one reactive monomer may be present in an amount between 10% and 40%, between 25% and 37%, between 28% and 35% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive monomer may be present in an amount between 10% and 40%, between 25% and 37%, between 28% and 35% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The sum of the at least one hydrophobic fumed silica and the at least one hydrophilic fumed silica may range from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive monomer may be present in an amount between 10% and 40%, between 25% and 37%, between 28% and 35% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 1.75% and 3.0% by weight of the total weight of the cosmetic composition, the sum of the at least one hydrophobic fumed silica and the at least one hydrophilic fumed silica may range from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive monomer may be present in an amount between 10% and 40%, between 25% and 37%, between 28% and 35% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate.

In the cosmetic composition within the brush-in bottle of the present disclosure, the at least one reactive oligomer may be present in an amount between 40% and 60%, between 42% and 53%, between 45% and 49% by weight of the total weight of the cosmetic composition. The at least one reactive monomer may be present in an amount between 10% and 40%, between 25% and 37%, between 28% and 35% by weight of the total weight of the cosmetic composition, and the at least one reactive oligomer may be present in an amount between 40% and 60%, between 42% and 53%, between 45% and 49% by weight of the total weight of the cosmetic composition. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive oligomer may be present in an amount between 40% and 60%, between 42% and 53%, between 45% and 49% by weight of the total weight of the cosmetic composition. The at least one reactive monomer may be present in an amount between 10% and 40%, between 25% and 37%, between 28% and 35% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive oligomer may be present in an amount between 40% and 60%, between 42% and 53%, between 45% and 49% by weight of the total weight of the cosmetic composition. The sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive oligomer may be present in an amount between 40% and 60%, between 42% and 53%, between 45% and 49% by weight of the total weight of the cosmetic composition. The at least one reactive monomer may be present in an amount between 10% and 40%, between 25% and 37%, between 28% and 35% by weight of the total weight of the cosmetic composition, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive oligomer may be present in an amount between 40% and 60%, between 42% and 53%, between 45% and 49% by weight of the total weight of the cosmetic composition. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive oligomer may be present in an amount between 40% and 60%, between 42% and 53%, between 45% and 49% by weight of the total weight of the cosmetic composition. The at least one reactive monomer may be present in an amount between 10% and 40%, between 25% and 37%, between 28% and 35% by weight of the total weight of the cosmetic composition, the at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive oligomer may be present in an amount between 40% and 60%, between 42% and 53%, between 45% and 49% by weight of the total weight of the cosmetic composition.

In the cosmetic composition within the brush-in bottle of the present disclosure, the at least one reactive oligomer may be present in an amount between 40% and 60%, between 42% and 53%, between 45% and 49% by weight of the total weight of the cosmetic composition. The at least one reactive monomer may be present in an amount between 10% and 40%, between 25% and 37%, between 28% and 35% by weight of the total weight of the cosmetic composition, and the at least one reactive oligomer may be present in an amount between 40% and 60%, between 42% and 53%, between 45% and 49% by weight of the total weight of the cosmetic composition. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive oligomer may be present in an amount between 40% and 60%, between 42% and 53%, between 45% and 49% by weight of the total weight of the cosmetic composition. The at least one reactive monomer may be present in an amount between 10% and 40%, between 25% and 37%, between 28% and 35% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive oligomer may be present in an amount between 40% and 60%, between 42% and 53%, between 45% and 49% by weight of the total weight of the cosmetic composition. The sum of the at least one hydrophobic fumed silica and the at least one hydrophilic fumed silica may range from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive oligomer may be present in an amount between 40% and 60%, between 42% and 53%, between 45% and 49% by weight of the total weight of the cosmetic composition. The at least one reactive monomer may be present in an amount between 10% and 40%, between 25% and 37%, between 28% and 35% by weight of the total weight of the cosmetic composition, the sum of the at least one hydrophobic fumed silica and the at least one hydrophilic fumed silica may range from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive oligomer may be present in an amount between 40% and 60%, between 42% and 53%, between 45% and 49% by weight of the total weight of the cosmetic composition. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 1.75% and 3.0% by weight of the total weight of the cosmetic composition, the sum of the at least one hydrophobic fumed silica and the at least one hydrophilic fumed silica may range from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive oligomer may be present in an amount between 40% and 60%, between 42% and 53%, between 45% and 49% by weight of the total weight of the cosmetic composition. The at least one reactive monomer may be present in an amount between 10% and 40%, between 25% and 37%, between 28% and 35% by weight of the total weight of the cosmetic composition, the at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 1.75% and 3.0% by weight of the total weight of the cosmetic composition, the sum of the at least one hydrophobic fumed silica and the at least one hydrophilic fumed silica may range from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, and the at least one reactive oligomer may be present in an amount between 40% and 60%, between 42% and 53%, between 45% and 49% by weight of the total weight of the cosmetic composition.

In the cosmetic composition within the brush-in bottle of the present disclosure, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one reactive monomer may be present in an amount between 10% and 40%, between 25% and 37%, between 28% and 35% by weight of the total weight of the cosmetic composition, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one reactive monomer may be present in an amount between 10% and 40%, between 25% and 37%, between 28% and 35% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one reactive monomer may be present in an amount between 10% and 40%, between 25% and 37%, between 28% and 35% by weight of the total weight of the cosmetic composition, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one reactive monomer may be present in an amount between 10% and 40%, between 25% and 37%, between 28% and 35% by weight of the total weight of the cosmetic composition, the at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 0.3% and 8%, between 0.75% and 6.5%, between 1.0% and 5.0%, between 1.5% and 4.0%, between 1.75% and 3.0% by weight of the total weight of the cosmetic composition, the sum of the at least one hydrophobic fumed silica and the at least one hydrophilic fumed silica may range from 0.4% to 10%, from 0.80% to 8.0%, from 1.2% to 6.5%, from 2% to 5%, from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate.

In the cosmetic composition within the brush-in bottle of the present disclosure, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one reactive monomer may be present in an amount between 10% and 40%, between 25% and 37%, between 28% and 35% by weight of the total weight of the cosmetic composition, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one reactive monomer may be present in an amount between 10% and 40%, between 25% and 37%, between 28% and 35% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The sum of the at least one hydrophobic fumed silica and the at least one hydrophilic fumed silica may range from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one reactive monomer may be present in an amount between 10% and 40%, between 25% and 37%, between 28% and 35% by weight of the total weight of the cosmetic composition, the sum of the at least one hydrophobic fumed silica and the at least one hydrophilic fumed silica may range from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 1.75% and 3.0% by weight of the total weight of the cosmetic composition, the sum of the at least one hydrophobic fumed silica and the at least one hydrophilic fumed silica may range from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate. The at least one reactive monomer may be present in an amount between 10% and 40%, between 25% and 37%, between 28% and 35% by weight of the total weight of the cosmetic composition, the at least one hydrophobic fumed silica may be present in an amount between 0.1% and 6%, between 0.2% and 4.0%, between 0.3% and 2.5%, between 0.4% and 2.0% by weight of the total weight of the cosmetic composition, the at least one hydrophilic fumed silica may be present in an amount between 1.75% and 3.0% by weight of the total weight of the cosmetic composition, the sum of the at least one hydrophobic fumed silica and the at least one hydrophilic fumed silica may range from 2.2% to 4.2% by weight of the total weight of the cosmetic composition, the ratio by weight of the at least one hydrophobic fumed silica to the at least one hydrophilic fumed silica may range from 0.05:1 to 1.5:1, from 0.08:1 to 1:1, from 0.15:1 to 0.80:1, from 0.20:1 to 0.70:1, the at least one reactive oligomer may be present in an amount between 40% and 60%, between 42% and 53%, between 45% and 49% by weight of the total weight of the cosmetic composition, and the hydrophobic fumed silica may have the INCI name Silica Dimethicone Silylate, Silica Dimethyl Silylate, Silica Silylate, Silica Cetyl Silylate or Silica Capryl Silylate.

In the cosmetic composition within the brush-in bottle of the present disclosure, the at least one photoinitiator may be present in an amount between 0.001% and 5%, between 0.01% and 2.5% by weight of the total weight of the cosmetic composition.

The cosmetic composition within the brush-in bottle of the present disclosure that may comprise at least one reactive monomer, at least one reactive oligomer, and at least one photoinitiator may optionally further comprise at least one additional cosmetic ingredient selected from the group of film formers, colorants, pigments, plasticizers, and light stabilizers. The at least one additional cosmetic ingredient may be any additional cosmetic ingredient, which may be present in any amount, as described above for the cosmetic composition.

Except in the Examples, or where otherwise explicitly indicated, all numerical quantities in this description specifying amounts of materials, application conditions, and the like, are to be understood as approximated, i.e., subject to a variability of plus or minus 5 percent, plus or minus 3 percent, plus or minus 1 percent, plus or minus 0.1 percent, or plus or minus 0.01 percent over the indicated value. It is to be understood that the upper and lower amount, range, and ratio limits set forth herein may be independently combined. Similarly, the ranges and amounts for each element of the technology described herein can be used together with ranges or amounts for any of the other elements.

### EXAMPLES

The following examples are included for illustrative purposes only and should not be construed as limitations on the invention claimed herein.

### Example 1—Cosmetic Compositions

The following Compositions 1 to 3 (see Table 1) were prepared by combining stabilizers and reactive monomers to make a stabilizer solution, combining film formers, reactive monomers and/or oligomers to make a film forming paste, combining silicas, reactive monomers and/or oligomers to make a silica paste, combining colorants and reactive monomers to form a dye solution, and combining photoinitiators and reactive monomers to form a photoinitiator solution. The stabilizer solution and reactive oligomers were mixed until homogenous to form Part 1. Then Part 1 and the film forming paste were mixed until homogenous to make Part 2. Then Part 2 and the silica paste were mixed until homogenous to make Part 3. Then Part 3, the dye solution, and the photoinitiator solution were mixed until homogenous to form a composition.

TABLE 1

| Ingredients | Composition 1 | Composition 2 | Composition 3 |
|---|---|---|---|
| (INCI names) | (% by weight) | (% by weight) | (% by weight) |
| Reactive Monomers: Tetrahydrofurfuryl | 32.5 | 32.4 | 33.1 |
| Methacrylate, Hydroxypropyl | | | |
| Methacrylate, PPG-5 Methacrylate, | | | |
| Triethylene Glycol Dimethacrylate | | | |
| Reactive Oligomers: Bis-HEMA | 46.9 | 46.7 | 47.6 |
| Poly(l,4-Butanediol)-22/IPDI | | | |
| Copolymer, Bis-HEMA | | | |
| Poly(Caprolactone Neopentyl | | | |
| Glycol)/IPDI Copolymer, Bis-HEMA | | | |
| Poly(Caprolactone/Pentaerythritol)/IPDI | | | |
| Copolymer Succinate, | | | |
| Isopropylidenediphenol PEG-2 | | | |
| Dimethacrylate, Isopropylidenediphenyl | | | |
| Bisoxyhydroxypropyl | | | |
| Methacrylate/TMDI Copolymer, | | | |
| Isopropylidenediphenyl | | | |
| Bisoxyhydroxypropyl Methacrylate | | | |
| Cellulose Acetate Butyrate | 14.49 | 14.42 | 14.73 |
| Trimethylbenzoyl Diphenylphosphine | q.s. | q.s. | q.s. |
| Oxide, Bis-Trimethylbenzoyl | | | |
| Phenylphosphine Oxide | | | |
| Ext. D&C Violet No. 2 | q.s. | q.s. | q.s. |
| BHT | q.s. | q.s. | q.s. |
| Silica Dimethicone Silylate | 1.64 | 0.82 | 0.45 |
| Silica | 2.46 | 3.68 | 2.07 |

q.s.: quantum sufficit, amount which is enough

### Example 2—Performance of Compositions: Sag Resistance

Compositions 1-3 were prepared as described in Example 1. Reference Compositions A-C were sculpting gels that do not include silica. All compositions were tested for their sag resistance according to the following procedure. A sheet of 0.5 cm graph paper was covered with glass. A 0.1 g droplet of each composition was placed on the glass and its location was recorded. The glass-covered paper was placed at a 45° angle and then heated in a 45° C. oven for 2 minutes. The glass-covered paper was returned to a horizontal orientation and the location of the terminal end of the droplet was noted. The distance between the pre- and post-heating locations was measured and was reported as distance traveled (cm). Less distance traveled indicates higher sag resistance. Results for Compositions 1-3 and Reference Compositions A-C are presented in Table 2.

TABLE 2

| Composition | Distance Traveled (cm) |
|---|---|
| 1 | 0.2 |
| 2 | 0.1 |
| 3 | 0.9 |
| Reference A | 1.6 |
| Reference B | 1.9 |
| Reference C | 2.7 |

The results shown in Table 2 demonstrate that Compositions 1-3 travel less than 1 cm after heating for 2 minutes at 45° C. In contrast, Reference Compositions A-C traveled more than 1.5 cm. The results demonstrate that the presently disclosed compositions have high sag resistance and do not run. Compared to sculpting gels lacking silica, the disclosed compositions have a higher sag resistance and less runniness.

### Example 3—Performance of Compositions: Leveling Test

Leveling is a measure of a composition's ability to flow out after application so as to obliterate any surface irregularities that may have been produced by the mechanical process of application. Compositions 1-3 were prepared as described in Example 1 and were subjected to a leveling test according to the procedure of ASTM D4062-11 with the following modifications. Briefly, equal amounts of the compositions were applied to a horizontal surface. Each composition was drawn down with a U-shaped film applicator having a shallow gap cut into one edge, such as the NYPC Leveling Test Blade (BYK-Chemie USA, Wallingford, Conn.). Into the shallow gap was cut a series of pairs of narrow notches having increasingly greater clearances (e.g., 10 mil, 20 mil, 40 mil; 0.01 in, 0.02 in, 0.04 in, respectively). Each pair of notches was separated by about 0.5 mil. The applicator was kept flat on the horizontal surface as it was drawn through the compositions. Each pair of resulting lines was monitored for the time at which the lines merged or leveled out. Time to level out, up to 45 minutes, was recorded. Results are presented in Table 3. Lower (faster) times indicate compositions with lower viscosity or more flow. Higher (slower) times indicate compositions with higher viscosity or less flow.

| | Time to Level Out (MM:SS) | | |
|---|---|---|---|
| | Composition 1 | Composition 2 | Composition 3 |
| 10 mil notch | 4:29 | >45:00 | 3:15 |
| 20 mil notch | 5:48 | >45:00 | 4:13 |
| 40 mil notch | >45:00 | >45:00 | 5:25 |

The results in Table 3 demonstrate that the time for Composition 3 to level out increased as the notch clearance increased. Composition 3 self-leveled at all notch clearances in less than five and a half minutes. Composition 1 took longer to level out than did Composition 3 at each notch clearance. Composition 1, after passing through a notch pair with a 40 mil clearance, did not self-level within the 45-minute testing period. Composition 2, at any tested notch clearance, did not self-level within the 45-minute testing period. Compositions that self-level are relatively easy to sculpt, and to sculpt quickly, into a nail enhancement. Compositions that do not self-level require manipulation, such as with a brush, to level out and form a nail enhancement.

1. A cosmetic composition comprising at least one hydrophobic fumed silica and at least one hydrophilic fumed silica, wherein the cosmetic composition is a gel sculpting nail composition.

2. The cosmetic composition of claim 1, wherein the ratio by weight of the at least one hydrophobic fumed silica to the at least one hydrophilic fumed silica ranges from 0.05:1 to 1.5:1.

3. The cosmetic composition of claim 1, wherein the at least one hydrophobic fumed silica is present in an amount between 0.1% and 6% by weight of the total weight of the cosmetic composition.

4. The cosmetic composition of claim 1, wherein the at least one hydrophilic fumed silica is present in an amount between 0.3% and 8% by weight of the total weight of the cosmetic composition.

5. The cosmetic composition of claim 1, wherein the sum of the at least one hydrophobic fumed silica and the at least one hydrophilic fumed silica ranges from 0.4% to 10% by weight of the total weight of the cosmetic composition.

6. The cosmetic composition of claim 1, wherein the composition comprises at least one reactive monomer, at least one reactive oligomer, and at least one photoinitiator.

7. The cosmetic composition of claim 6, wherein the at least one reactive monomer is present in an amount between 10% and 40% by weight of the total weight of the cosmetic composition.

8. The cosmetic composition of claim 6, wherein the at least one reactive oligomer is present in an amount between 40% and 60% by weight of the total weight of the cosmetic composition.

9. The cosmetic composition of claim 6, wherein the composition comprises at least one additional cosmetic ingredient selected from the group consisting of film formers, colorants, pigments, plasticizers, and light stabilizers.

10. A method of sculpting an artificial nail onto at least a portion of a nail, the method comprising:
applying onto at least a portion of the nail a layer of a cosmetic composition comprising at least one hydrophobic fumed silica and at least one hydrophilic fumed silica, wherein the cosmetic composition is a gel;
sculpting the cosmetic composition into a desired shape and length; and
curing the cosmetic composition.

11. The method of claim 10, wherein the applying, sculpting, and curing of the cosmetic composition are repeated at least once per nail.

12. The method of claim 10, wherein the curing of the cosmetic composition is performed with a visible or UV nail lamp.

13. The method of claim 10, wherein the curing of the cosmetic composition lasts for at least 10 seconds.

14. The method of claim 10, wherein the layer of the cosmetic composition has a thickness of at least 1 mm.

15. A brush-in bottle containing a cosmetic composition comprising at least one hydrophobic fumed silica and at least one hydrophilic fumed silica, wherein the cosmetic composition is a gel sculpting nail composition applied or applicable to at least a portion of a nail with the brush.
